(19) United States
(12) Patent Application Publication
(10) Pub. No.: US 2010/0185222 A1
(43) Pub. Date: Jul. 22, 2010

(54) Vacuum-Assisted Microscale Cutting Device

(75) Inventor: Christopher Guild Keller, El Cerrito, CA (US)

Correspondence Address: FENWICK & WEST LLP, SILICON VALLEY CENTER, 801 CALIFORNIA STREET, MOUNTAIN VIEW, CA, 94041, US

(73) Assignee: Mynosys Cellular Devices, Inc., Albany, CA (US)

(21) Appl. No.: 12/377,228

(22) PCT Filed: Aug. 13, 2007
(86) PCT No.: PCT/US07/75840

§ 371 Date: Feb. 11, 2009

Related U.S. Application Data
(60) Provisional application No. 60/837,401, filed on Aug. 11, 2006.

(51) Int. Cl. A61B 17/32; A61B 17/3211; A61M 5/178

(52) U.S. Cl. 606/170; 606/172; 604/164.01

(57) ABSTRACT

A vacuum-assisted microscale cutting instrument applies a vacuum pressure to pull an area of tissue towards a microknife. The cutting instrument can be configured to make one or more stabbing cuts or a slicing cut, and the housing of the instrument is shaped to address any of a variety of tissue geometries to allow a vacuum seal to be created therewith. To achieve a consistent cut depth in the tissue, a depth stop may be used to prevent the knife from cutting deeper than a predetermined depth.

### CROSS REFERENCE TO RELATED APPLICATIONS

This application claims the benefit of U.S. Provisional Application No. 60/837,401, filed Aug. 11, 2006, which is incorporated by reference in its entirety.

### BACKGROUND

This invention relates generally to microscale cutting instruments and techniques for their operation.

In microsurgery it is often necessary to make precise incisions in tissue structures on a very small scale. Generally, a handheld scalpel is used, and for the finest work a surgeon may also look through a stereo microscope when making precise cuts. The traditional handheld scalpel and other traditional cutting systems rely on the resilience of the surrounding tissue to pull back and provide the reaction force necessary to oppose the cutting force rather than merely deflecting in response to the force of the cutting instrument. But on very small scales, the deflection of the tissue caused by the cutting force can be very significant, and the deflection manifests, in large part, in the stretching of the tissue.

A surgeon may try to decrease the cutting force applied to the tissue by making many repetitive shallow cuts, repeating the same cut over and over again. One problem with this approach, however, is that even with a very sharp knife the cutting force is still sufficient to distort tissues on a scale larger than the size of the desired cut. Many tissues in the body, such as nerves and blood vessels are easily stretched for short distances as they must be to accommodate the normal movement of the body in daily life. Therefore, even with very small forces applied to tissue, a significant amount of stretching can occur.

Existing surgical knives that address this issue include the ultrasonic knife. The ultrasonic knife uses the inertia of the tissue to oppose the knife's force and hold it in place as the knife cuts. For this to work, the knife has to move very fast and make many short cuts per second. The consequences of this are that a lot of energy is dissipated in the tissue as heat, and since many small cuts are made, more damage is done to the tissue on the microscale.

What is needed are techniques and devices for making small, precise cuts in tissue, or other material in which a microscale incision is desired, without applying significant cutting forces that cause undesirable stretching of the tissue being treated.

### SUMMARY

Embodiments of the invention include vacuum-assisted microscale cutting instruments. In operation, the instrument applies a vacuum to an area of tissue where a cut is to be made, where the vacuum pressure applied to the tissue pulls the tissue towards the knife while the knife is pressed against the tissue. This provides at least a portion of the reaction force needed for cutting to occur, enabling precise cutting into materials that cannot by themselves provide the reaction force needed for the cutting. By pulling the tissue into the knife and keeping the force circuit within the device and adjacent tissue, gross stretching of tissue a distance away from the cut can be avoided. This technique enables efficient precise cutting of small tissue structures in microsurgery and other types of materials in non-medical applications. For a given device design and a given tissue, the depth, length, and width of cutting can be made to be more reproducible, helping to lessen the skill of the surgeon as a variable in the cutting process. Embodiments of the invention thus allow cutting operations to be done faster, more precisely, and without a high degree of operator skill.

Different embodiments of the microscale cutting instrument may include various mechanical elements. For example, a depth stop may be mounted to the housing to prevent cuts beyond a predetermined depth. Moreover, the microknife may be mounted in a housing that is detachable from a handle assembly, so that a cutting head portion of the device may be removed and disposed of after use or interchanged for a different procedure, and the handle assembly can be reused.

In operation, according to one embodiment, an operator places the microscale cutting device against an area of tissue to be treated, which creates a vacuum seal with the tissue. The operator then turns on a vacuum source to reduce the pressure within the housing of the device relative to the atmosphere. This reduced pressure tends to cause a force in the tissue upward toward the housing, and the pressure may also cause one or more pneumatic actuators coupled to the microknife to move so that the knife moves as well. These one or more actions cause the microknife to cut into the tissue in a precise and repeatable manner, configured according to the particular design of the cutting device.

Various configurations of the device can be used to make different cuts. For example, the device may be configured to make a stab incision with stationary knife or with a knife that moves straight into the tissue. Alternatively, the device can create a slice cut, where the knife moves into the tissue and also in a direction transverse to it to make an incision longer than the width of the knife's cutting edge. The knife may also be curved to cut a strip of the tissue.

In one embodiment, surgical tools other than a microknife are used in the cutting device. For example, a needle may be mounted within the housing of the device in various embodiments described herein for the microknife. Rather than make a knife cut, actuation of such a device results in a precise injection, which may deliver a medicine or other injectable agent or other biological material, such as DNA, proteins, and cells. The instrument may be shaped to enable injections in areas that are difficult to reach with conventional means, such as within an artery or vein.

Moreover, different embodiments of the device may be configured to address different tissue geometries. For example, the device may include a housing to address planar, cylindrical, spherical, or other surface geometries so as to enable a vacuum with an area of the tissue surface. In one embodiment, the housing is shaped to fit within a tubular structure, such as an artery, and the knife is arranged to cut into a wall of the tubular structure. In this way, the device can be used for a number of different procedures and tissue areas where a microscale cut is desirable.

### BRIEF DESCRIPTION OF THE DRAWINGS

FIGS. 1A and 1B are cutaway side views of a vacuum-assisted microscale cutting device during operation, in accordance with an embodiment of the invention.

FIG. 2 illustrates a vacuum-assisted microscale cutting device adapted for a planar tissue surface, in accordance with an embodiment of the invention.

FIG. 3 illustrates a vacuum-assisted microscale cutting device adapted for a cylindrical tissue surface, in accordance with an embodiment of the invention.

FIG. 4 illustrates a vacuum-assisted microscale cutting device adapted for a convex spherical tissue surface, in accordance with an embodiment of the invention.

FIG. 5 illustrates a vacuum-assisted microscale cutting device adapted for a concave spherical tissue surface, in accordance with an embodiment of the invention.

FIGS. 6A through 6C illustrate a vacuum-assisted microscale cutting device adapted for cutting the inside of a cylindrical surface, in accordance with an embodiment of the invention.

FIG. 7 illustrates a system for performing vacuum-assisted microscale incisions, in accordance with an embodiment of the invention.

FIG. 8 shows a system for performing vacuum-assisted microscale incisions with a control subsystem, in accordance with an embodiment of the invention.

FIGS. 9A through 9D illustrate a process for cutting tissue, in accordance with an embodiment of the invention.

FIGS. 10A through 10D illustrate a process for performing a vacuum-assisted injection, in accordance with an embodiment of the invention

FIG. 11 is an exploded view of an assembly of a portion of a microscale cutting device, in accordance with an embodiment of the invention.

FIG. 12 is a side view of a portion of a microscale cutting device having a moving blade, in accordance with an embodiment of the invention.

FIGS. 13A through 13E illustrate a process for cutting tissue by making a series of stab cuts in tissue, in accordance with an embodiment of the invention.

FIGS. 14A through 14D illustrate a process for cutting tissue by making a slicing cut through tissue, in accordance with an embodiment of the invention.

FIGS. 15A and 15B illustrate a process for cutting a strip of tissue using a three-dimensional blade, in accordance with an embodiment of the invention.

FIGS. 16A and 16B illustrates a vacuum-assisted microscale cutting device having a compliant structure, in accordance with an embodiment of the invention.

FIG. 17 illustrates a system for performing vacuum-assisted microscale incisions, in accordance with an embodiment of the invention.

FIGS. 18A through 18C illustrate a process for cutting with the system of FIG. 17, in accordance with an embodiment of the invention.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

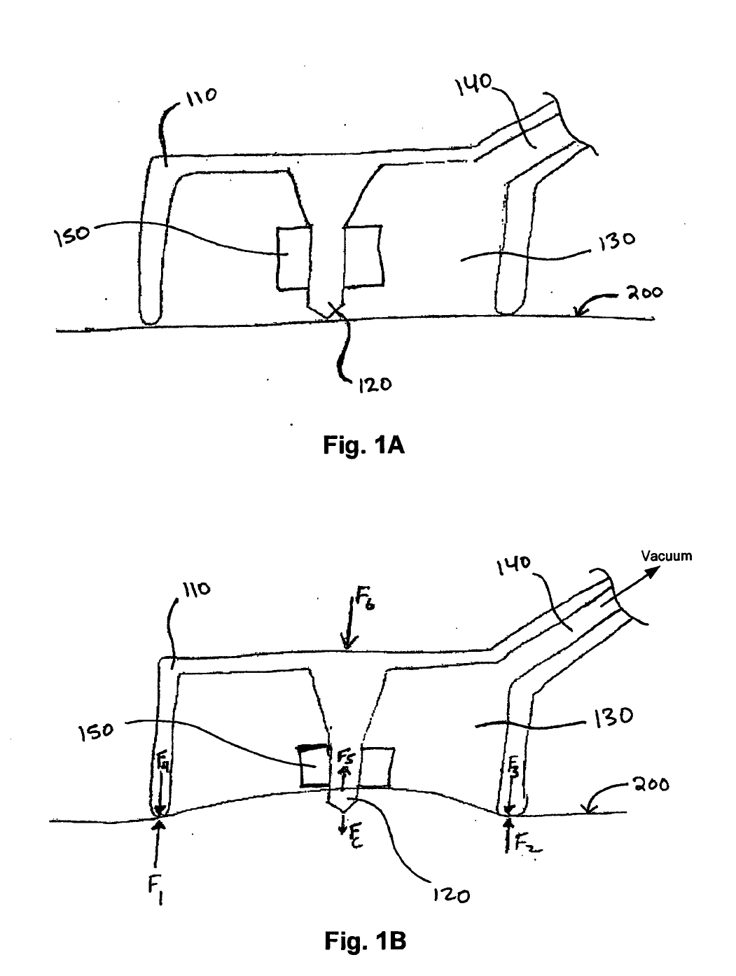

FIG. 1A shows a cross section of an embodiment of a vacuum-assisted microscale cutting device. The device comprises a housing 110, a vacuum connector 140, and a microknife 120 mounted within the housing 110. The housing 110 of the device is designed to fit against an area of tissue 200 where a microscale cut is to be made. In particular, the housing 110 is designed so that a vacuum seal can be made between the tissue 200 and the housing 110. For purposes of certain embodiments of the invention, a perfect vacuum seal is not necessary.

The interior of the housing 110 forms a vacuum chamber 130, which is coupled to the vacuum connector 140. The vacuum connector can be coupled to a vacuum source to remove air from the chamber 130, thereby reducing the pressure within the chamber 130 when a seal is made between the housing 110 and tissue. When no vacuum is applied, as shown in FIG. 1A, the microscale cutting device confronts undisturbed tissue.

FIG. 1B illustrates the device of FIG. 1A where a vacuum source is coupled to the vacuum connector 140 and activated. Because the pressure within the chamber 130 is reduced relative to the atmospheric pressure that exists outside the device and within the tissue 200, the surface of the tissue 200 that falls within the area covered by the evacuated chamber 130 moves vertically up. As shown, the surface of the tissue 200 moves to a height that is above the cutting edge of the microknife 120, which causes a corresponding cut in the tissue 200. In this way, the tissue 200 is simply pulled up into the cutting edge of the knife 120. The convex curvature puts the surface of the tissue 200 in tension, which also helps the cutting to occur. Very little or no stretching or distorting of the adjacent tissue occurs, and any uncertainty about whether the tissue was actually cut or how deep the cut was is greatly reduced or eliminated.

The forces involved in this process are illustrated in FIG. 1B. The pressure of the atmosphere results in a force F₆ on the top of the housing 110 that pushes it against the tissue 200, which tends to keep the device in place. The force from the operator pushing the device against the tissue may also contribute to F₆, but once the vacuum valve is opened, the operator can reduce his force to zero. Contact forces F₄ and F₃ are balanced by the opposing reaction forces F₁ and F₂. The forces at the edge of the microknife 120, cutting force F_{c} and the associated reaction force F₅, are less than F₆, so the device is not disturbed from its position during the cutting process. When the vacuum is applied, the tissue 200 will move up until the elastic restoring force in the tissue 200 balances the force caused by the pressure differential. The height at which this mechanical equilibrium is reached depends on the length and width of the chamber 130 and the elastic stiffness of the tissue 200. Atmospheric pressure provides a force of 0.1 N/mm². In a typical embodiment, a pressure within the chamber 130 in the range of about 4 to about 400 Torr is sufficient to cause the tissue 200 to rise and produce a successful cut.

In one embodiment, the device further comprises a cutting depth stop 150 coupled or otherwise fixed relative to the knife 120. The depth stop 150 is configured to prevent the knife 120 from cutting into tissue beyond a predetermined depth. As shown in FIG. 1B, the depth stop 150 can enable an extremely precise cut since any sufficient vacuum within the chamber 130 should yield the maximum cut depth allowed by the depth stop 150. Beneficially, the depth stop 150 removes the dependency on the elastic stiffness of the tissue 200 in determining depth of cut. It also allows the device to used on different tissues and yield the same depth of cut. Moreover, the depth stop 150 may be adjustable to provide for variable cut depths, or it may be fixed for a single cut depth.

The microknife 120 may comprise a simple standalone blade, or it may comprise a blade integrated with a thicker silicon supporting structure. The knife blade may be made separately from a supporting structure and then attached to it to make a complete assembled system. In the case of a knife blade integrated with a supporting silicon structure, handling of the knife blade for assembly is easier since is the part is bigger. A simple knife blade may also be glued into a cavity mold to mount it within the device.

In the case of a simple standalone blade, the device may be made by microinjection molding of a transparent plastic (such as polycarbonate) to form the housing 110. The microknife 120 is then glued or otherwise attached into a small cavity in the housing 110 that has been molded for it in the plastic. The cutting edge of the knife 120 is then clearly visible through the side of the transparent chamber, so any tissue to be cut can be seen clearly. For this purpose, the sides of the transparent plastic of the housing 110 are preferably smooth and flat to avoid distorting the image.

In one embodiment, the microknife 120 is self-sharpening. The microknife blade can be made to be self-sharpening by forming the knife of a thin layer of a relatively hard material (e.g., silicon nitride) an a support structure of a relatively soft material (e.g., silicon). When used to cut through a material, the softer support structure wears more quickly and exposes the harder material, which acts as the cutting edge of the knife. The sharpness of the microknife thus follows from the thickness of the harder material. For example, if the hard material is 100 angstroms thick, the cutting edge will not be more than 100 angstroms thick itself. Various methods for forming microscale cutting instruments that can be used with embodiments of the invention, including instruments having self-sharpening cutting edges, are disclosed in International Application No. PCT/US07/61701, filed Feb. 6, 2007, which is incorporated by reference in its entirety.

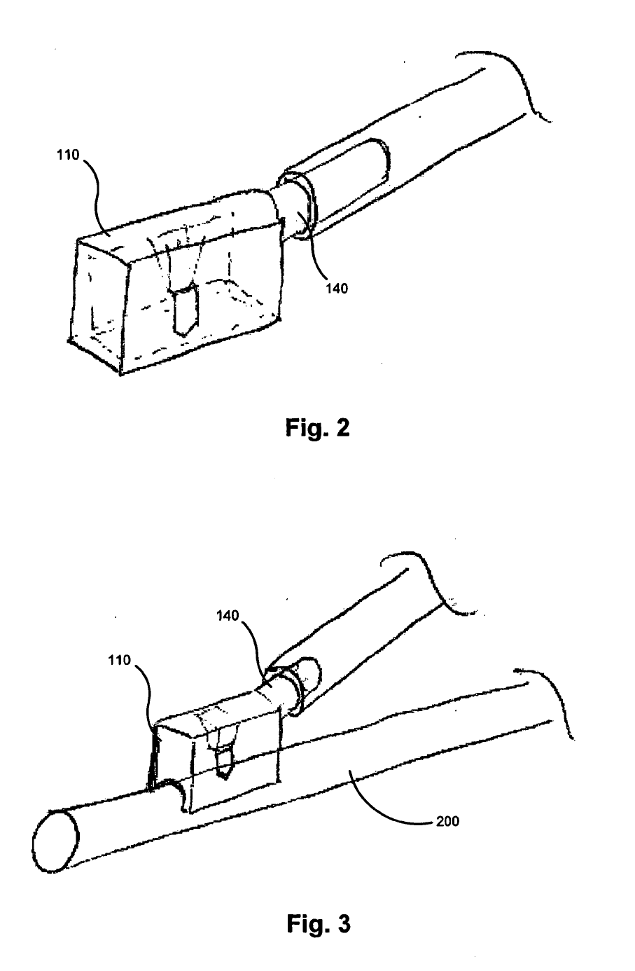

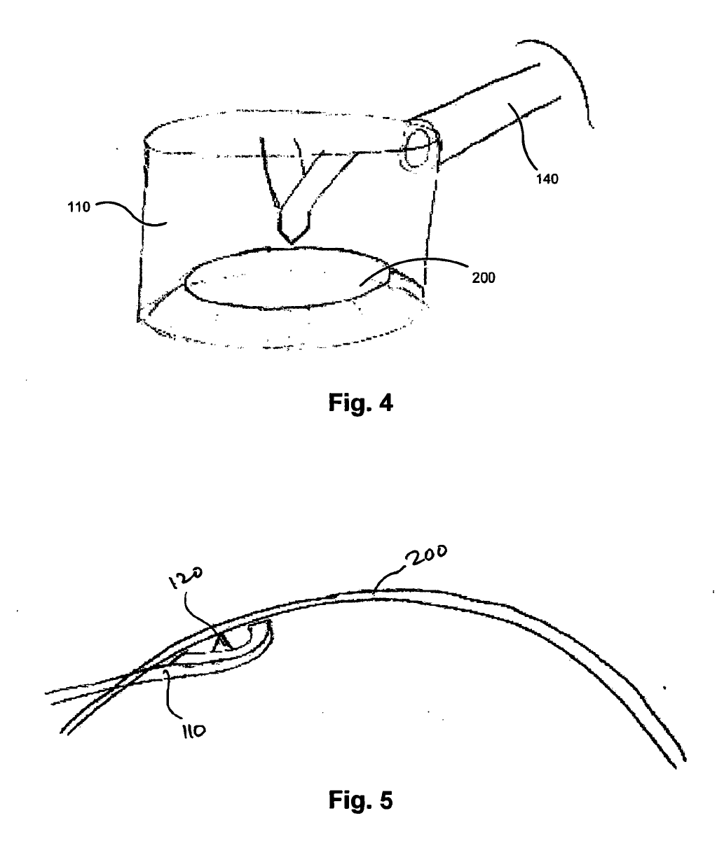

The housing 110 of the microscale device is shaped at its open end to conform to the geometry of tissue 200 with which the device is intended to be used. For example, FIG. 2 illustrates a device where the housing 110 is suitable for establishing a vacuum when confronting a tissue having a flat or planar surface. Alternatively, the housing 110 of the device shown in FIG. 3 is suitable for establishing a vacuum when confronting a tissue having a cylindrical surface with a particular radius of curvature. For example, a nerve having a radius of 1.5 mm could be addressed by this structure if the open end of the housing 110 is constructed with the same or similar radius. This device could then address any cylindrical tissue having a radius of about 1.5 mm. Whatever the radius of the tissue of interest, a device of the same radius could be made to address it by simply forming a correspondingly shaped housing 110. Similarly, FIG. 4 shows a device that has a housing 110 capable of addressing a is spherical surface, such as an eye or an egg cell. Conversely, FIG. 5 shows a device that has a housing 110 capable of addressing a addressing a concave spherical surface, such as the inner surface of a cornea. Housings to mate with irregularly shaped surfaces can also be constructed for other special purpose procedures. For any of these designs, the principle of closing the force circuit at the perimeter of the housing 110 applies, as discussed above in connection with FIGS. 1A and 1B.

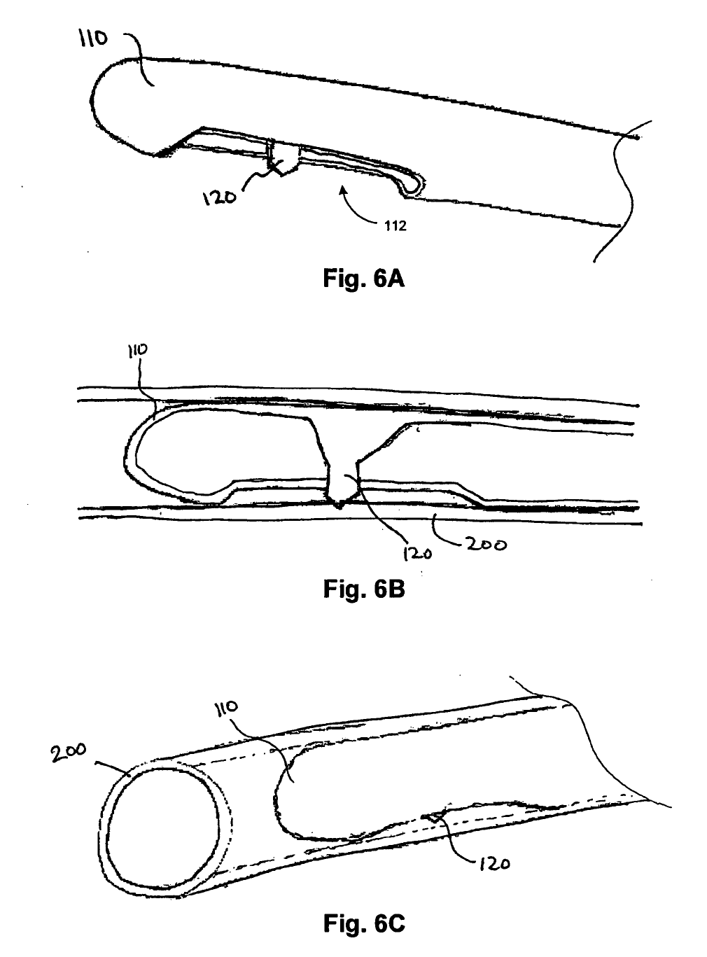

In yet another configuration, FIG. 6A shows a device that has a housing 110 capable of addressing concave cylindrical surface such as the inner surface of a vessel such as an artery. The tubular chamber within the housing 110 has an opening 112 to suck in the tissue and pull it into the knife 120 when a vacuum pressure is applied to the chamber. The tubular shape of the housing 110 enables it to be inserted into a cylindrical vessel, and remote operation may be possible by attaching the device to the end of a catheter. FIG. 6B shows a cutaway side view and FIG. 6C shows a perspective view of the device inserted into a tubular tissue structure 200, such as an artery. Once inserted, the vacuum can be applied so that the inside of the tissue wall deflects into and is therefore cut by the knife 120.

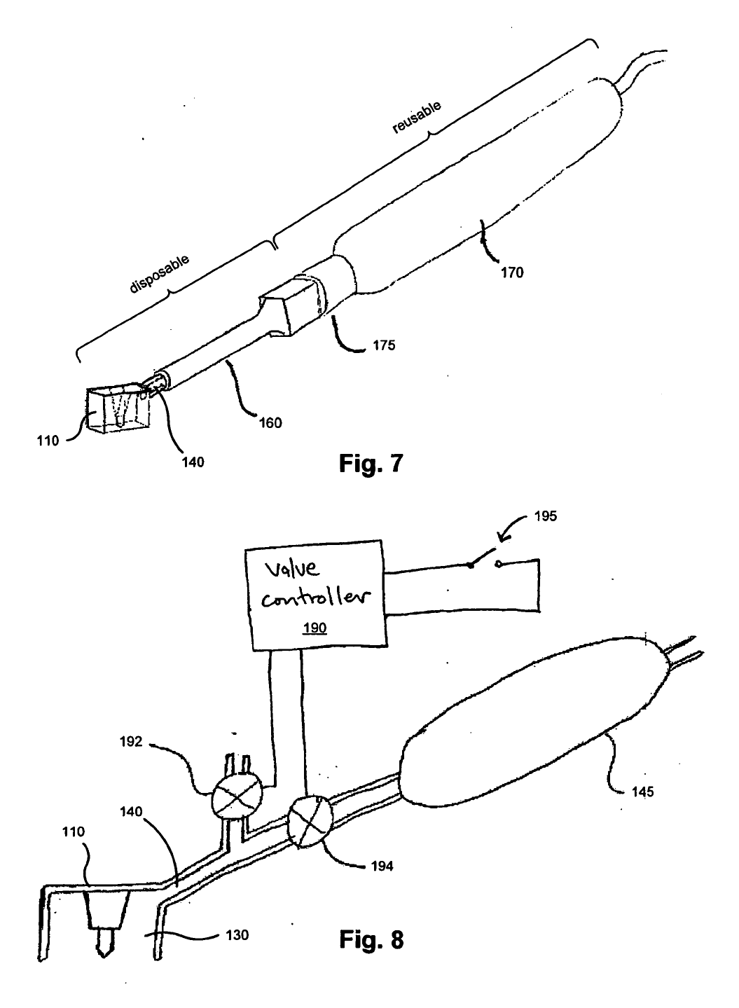

FIG. 7 illustrates an embodiment of a system for performing vacuum-assisted cutting in accordance with one embodiment. The system includes a disposable portion, which may comprise a housing 110, knife 120, and vacuum connector 140, such as discussed above. This disposable portion of the system can be coupled into a reusable portion of the system by the vacuum connector 140, so that the reusable portion of the system provides the vacuum source for actuation of the cutting device.

In one embodiment, the reusable portion of the system comprises tubing 160, a handle 170, and a connector 175 (such as a Luer lock) therebetween. In this embodiment, the tubing 160 comprises a blunt end hypodermic needle, which fits with the vacuum connector 140 of the disposable device by way of a tapered friction fit. A standard 3-degree taper fit may be used to produce a low leakage connection that can be conveniently connected and disconnected. This allows the disposable portion of the system to be removed and replaced easily. The tubing 160 preferably connects via a Luer lock connection 175 to the handle 170.

The handle 170 may contain a control valve that allows an operator to apply the vacuum to the chamber 130 or to release the vacuum pressure applied to the chamber 130. Alternatively, the control valve for the vacuum pressure may be located off the handle 170, where the handle 170 merely comprises a hollow tube that communicates the vacuum to the device. In one embodiment, a foot-operated switch is used as a convenient means for controlling actuation of the vacuum source and or control of the valve allowing the vacuum pressure to the chamber 130. Alternatively, the handle 170 may contain a miniature battery-powered vacuum pump with an on/off switch on the handle 170.

FIG. 8 illustrates a control system for a system for performing vacuum-assisted cutting in accordance with one embodiment. The vacuum port 140 of the vacuum-assisted cutting device is connected to two channels, as shown. One of the channels connects the device's chamber 130 to the atmosphere by an air valve 192, and the other channel connects the chamber 130 to a vacuum source 145 by a vacuum valve 194. Preferably, the valves 192 and 194 are located as close as possible to the chamber 130 to minimize the dead volume for the system. A valve controller is operably coupled to each of the valves 192 and 194, and the valve controller 190 can be operated to open and close each vale 192 and 194 independently. A switch 195 (such as a foot-operated pedal) may be used by an operator to control the valve controller 190.

In use, an operator places the open face of the device's chamber 130 in contact with an area of the tissue to be cut. The operator then activates the system, e.g., by stepping on a foot switch 195, which causes the valve controller to run its program. In one embodiment, the program comprises the steps: (1) close the air valve 192, (2) open the vacuum valve 194 for a predetermined time, (3) close the vacuum valve 194, and (4) open the air valve 192. The controller 190 may be programmed to keep cycling through the program until the switch 195 is depressed again (so that the switch 195 acts as an on/off switch). Different programs may be used for a different sequences of steps, as desired. Typically, there would be no use for opening both valves 192 and 194 at once, as the vacuum 145 would just pull in ambient air from the atmosphere through the open air valve 192.

One of the parameters that may be set by the system is the vacuum pressure applied to the chamber 130. Typically, the vacuum pressure would be in the range of about 4 to about 400 Torr, depending on the application. The difference between ambient air pressure and vacuum pressure, multiplied by the area of the chamber opening contacting the tissue, determines the force on the tissue. The lower the pressure in the chamber, the greater the force pulling on the tissue will be. The pressure should not go below about 4 Torr, since at that low of a pressure water at room temperature starts to boil.

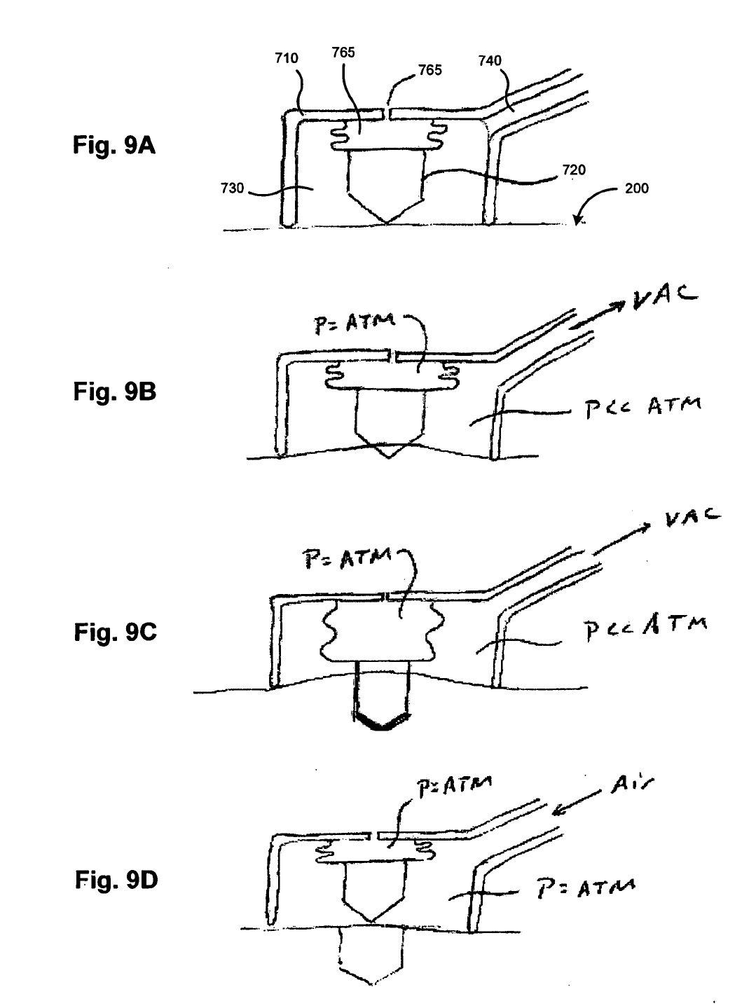

FIG. 9A shows a vacuum-assisted device for making a precise incision over an area of tissue. The device comprises a housing 710 with an opening shaped to fit over an area of tissue, where inside the housing 710 is a vacuum chamber 730. This chamber 730 is coupled to a vacuum port 740 for reducing the pressure in the chamber 730. As shown, the device includes a knife 720 for cutting into tissue; however, the device may alternatively contain a needle or any other type of cutting instrument for inserting the instrument into confronting tissue. The knife 720 is mounted to the housing 710 by a pneumatic actuator 760, which may be a bellows, diaphragm, piston, or any other suitable structure. The pneumatic actuator 760 is coupled to the ambient environment by a hole 765, which may be a pinhole, thereby keeping the steady state of the pneumatic actuator 760 at atmospheric pressure.

FIG. 9A shows the device at rest in atmospheric pressure. In operation, the device is placed over an area of tissue where an incision is desired. As shown in FIG. 9B, the vacuum is applied, and the pressure inside the chamber 730 lowers to a level that is much less than the ambient atmosphere. This applied vacuum in the chamber 730 causes the tissue to move up towards the knife 720. In addition, because the pneumatic actuator 760 is open to the atmosphere, it expands in the presence of the vacuum pressure, as shown in 9C. The knife 720, which is mounted on the pneumatic actuator 760, is thus forced downwards towards the tissue, which further assists in the cutting action. The rate at which the pneumatic actuator 760 expands can be controlled by the dimensions of the hole 765 that allows air flow from the atmosphere into and out of the pneumatic actuator 760. For the fastest cutting action, the hole 765 should be sufficiently large, and it can have the same diameter as the pneumatic actuator 760 itself. As shown in FIG. 9C, the pneumatic actuator 760 has reached mechanical equilibrium, and the deepest possible incision has been made. When finished with the cut, the vacuum source is turned off and the chamber 730 is vented. The bulged tissue and the knife 720 and pneumatic actuator 760 all return to their original position, as shown in FIG. 9D.

By causing actuation of the knife 720, this device may provide deeper cuts than might be possible by deflection of tissue alone. One application for a device according to this embodiment is cutting through the side of the cornea to perform cataract surgery. This cut should be self-sealing after the operation is over, which means the cut has to be very smooth and straight. But the gross distortions of the tissue geometry and the shakiness and random movements typically introduced by the surgeon make an ideal cut impossible. To address these factors that reduce the quality of the cut, this embodiment locks the tissue geometry (e.g., a convex spherical surface) by the matching geometry of the housing 710 (and thus the vacuum clamp), and the knife 720 is able to move straight in because the surgeon has been mechanically eliminated from the force circuit.

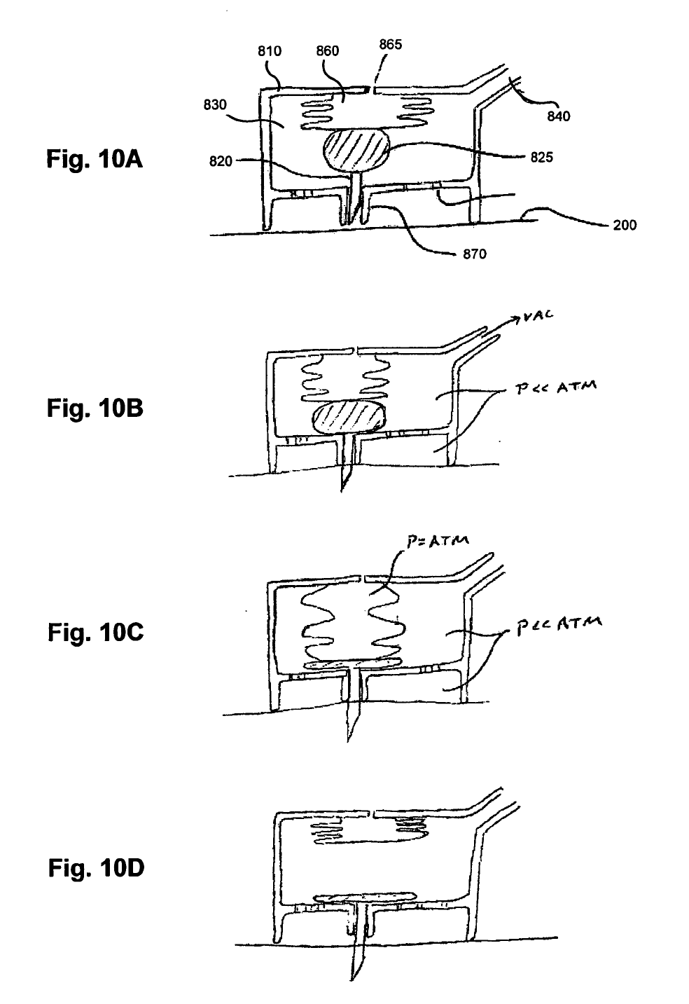

FIG. 10A shows a vacuum-assisted device for making a precise injection. The device comprises a housing 810 with an opening shaped to fit over an area of tissue and form a vacuum chamber 830 thereby. The chamber 830 is coupled to a vacuum port 740 for reducing the pressure in the chamber 730. A needle 820 is mounted to the housing 810 by a pneumatic actuator 860, which may be a bellows, diaphragm, piston, or any other suitable structure. The pneumatic actuator 860 is coupled to the ambient environment by a hole 865, thereby keeping the steady state of the pneumatic actuator 860 at atmospheric pressure. The needle 820 is held in position by a guiding collar 870 and is attached at one end to a liquid-filled capsule 825. The liquid-filled capsule 825 contains a liquid, such as a medicine or other therapeutic product or biological material, such as DNA, proteins, and cells, to be injected into the tissue (any of these materials collectively referred to herein as “therapeutic agents”). In one embodiment, the device is mounted within a catheter to allow injection into tissues not accessible to normal hypodermic needles.

FIG. 10A shows the device at rest in atmospheric pressure. In operation, the device is placed at an area of tissue where an injection is desired. As shown in FIG. 10B, the vacuum is then applied to the chamber 830. The vacuum causes the pressure within the chamber 830 to become much lower than the ambient pressure, which in turn causes air to flow into the pneumatic actuator 860. The rate at which the air can enter the pneumatic actuator 860 is controlled by the dimensions of the hole 865. As shown in the progression of FIGS. 10B and 10C, the pneumatic actuator 860 continues to expand. In addition to pressing the needle 820 into the tissue, this action eventually presses the liquid-filled capsule 825 against the guiding collar 870 or other part of the device housing 810. At some point, this causes a frangible seal of the liquid-filled capsule 825 to break at the orifice of the hollow needle 825, thereby allowing the liquid to flow from the capsule 825 through the needle 820 and into the tissue 200. Once enough time has passed for the injection to occur, the vacuum source is turned off and the chamber 830 is vented, as shown in FIG. 10D. The device can then be pulled away from the tissue 200, taking the needle 820 with it.

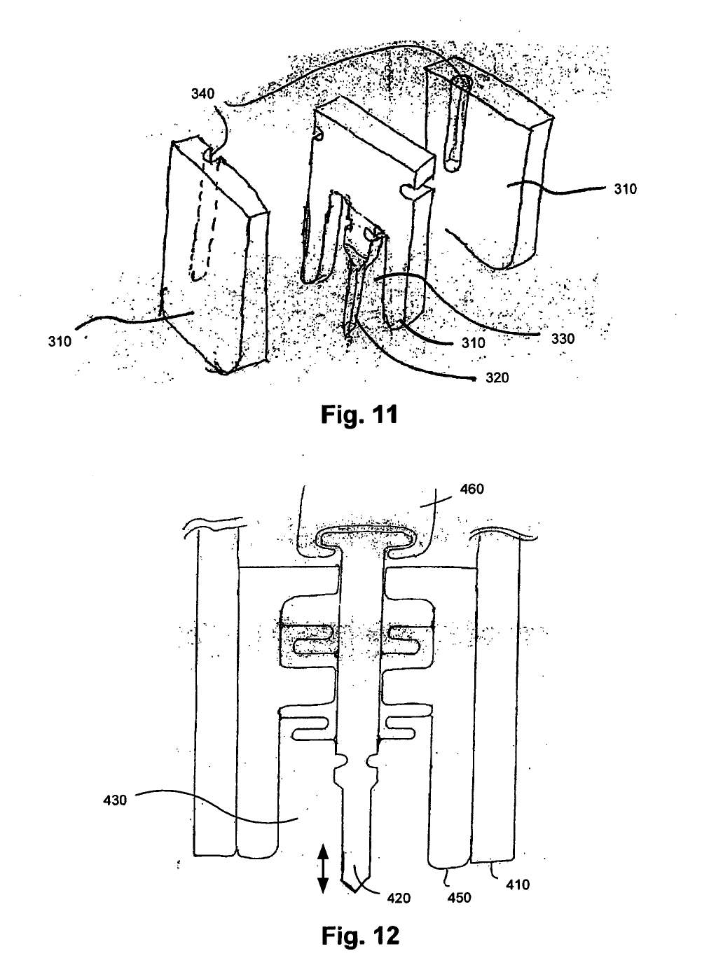

FIG. 11 is an exploded view of an alternative embodiment of the microscale cutting device. In this embodiment, the housing 310 of the device is formed by sandwiching together the three layers 310, as illustrated. A microknife 320 is fixed to the middle layer of the housing 310, where the knife 320 may be advantageously formed on an integral layer of silicon. The housing layers 310 are combined to enclose a vacuum chamber region 330, which can be placed over an area of tissue as described above. A vacuum channel 340 is formed in one or both of the outer layers of the housing 330 so that the vacuum channel 340 is in communication with the chamber 330. A vacuum source can be coupled to the vacuum channel 340 (e.g., using tubing, not shown) to provide the desired vacuum pressure within the chamber 330 needed for operation of the cutting instrument. Control of the vacuum actuation may be provided using various means, such as by blocking the vacuum channels 340 or controlling the vacuum pressure to the tubing using a valve.

Rather than keeping the knife stationary with respect to the housing of the device, in one embodiment the knife itself may move in the cutting direction. This action may be performed in addition to an applied vacuum pressure. In such an embodiment, a lower vacuum pressure may be used, since the cutting motion is created by application of a force to the knife as well as action on the tissue caused by the vacuum pressure of the chamber. An embodiment of a device for performing this technique is illustrated in FIG. 12.

FIG. 12 illustrates a device for performing microscale cutting, where the knife 420 is moved up and down into tissue which the housing 410 is advanced along the tissue. This embodiment also includes a vacuum chamber 430, as described above, to reduce the amount of deformation in the tissue resulting from the action of the knife 420. In contrast to the embodiments described above, in this embodiment the knife 420 is pushed into the tissue and then pulled out of it by an actuator 460. The actuator 460 may comprise a wire that is mechanically driven in cycles to cause the movement of the knife 420. As long as the device is moved at a rate that advances it one half of the width of the knife 420 or less during each cycle, the device can make a continuous cut in the tissue.

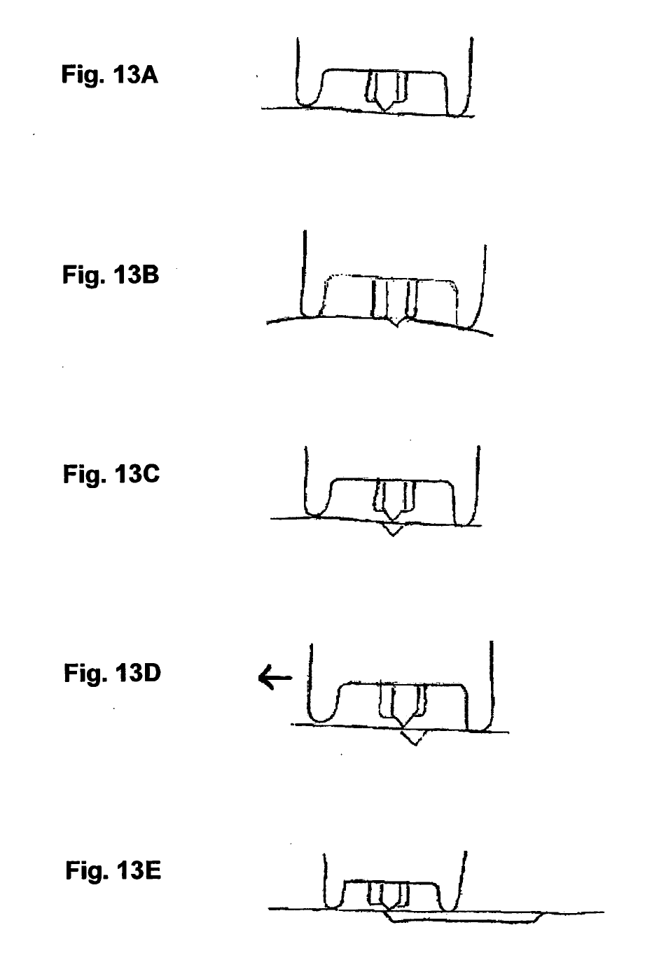

FIGS. 13A through 13E illustrate a sequence of steps for operating the device to perform microscale cutting, in accordance with one embodiment. In FIG. 13A, the microscale cutting device is brought to the surface of the tissue and placed against it. In FIG. 13B the vacuum pressure is applied to the device, and the surface of the tissue is pulled up to the depth stop. This action results in a stabbing incision into the tissue by the microknife. In FIG. 13C the vacuum is turned off, and the surface of the tissue pulls back to its original height.

In many microscale applications, this one stab incision produced after the step in FIG. 13C is all that is desired, so the cutting operation may be complete. However, if a longer incision is desired, the cutting instrument can then be moved along the tissue surface, as shown in FIG. 13D. In FIG. 13D the device has been translated in the desired direction of the incision by an incremental distance, which is preferably less than one half of the width of the knife blade. This maximum translation of the device enables a continuous incision to be made in the tissue. Once the device is moved the desired distance, the vacuum is again applied and then released (e.g., FIGS. 13B and 13C are repeated). This three-step cycle of cut, release, and move can be repeated until the incision reaches the desired length, as shown in FIG. 13E.

Depending on the details of the particular design, the vacuum can typically be turned on and off anywhere from about 10 to 100 times per second. The cycle rate may be configurable by the operator. The operator can set the cycle rate and then move the knife at a rate that advances it one half of the blade width or less during each cycle. This maximum speed can be easily calculated given the blade width and cycle rate.

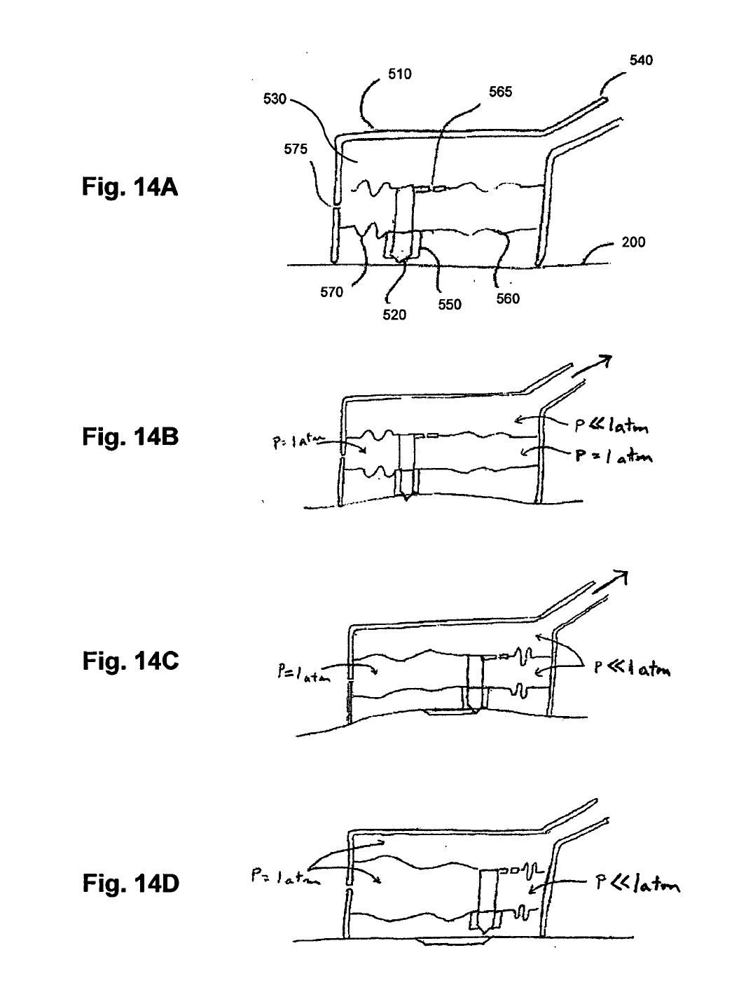

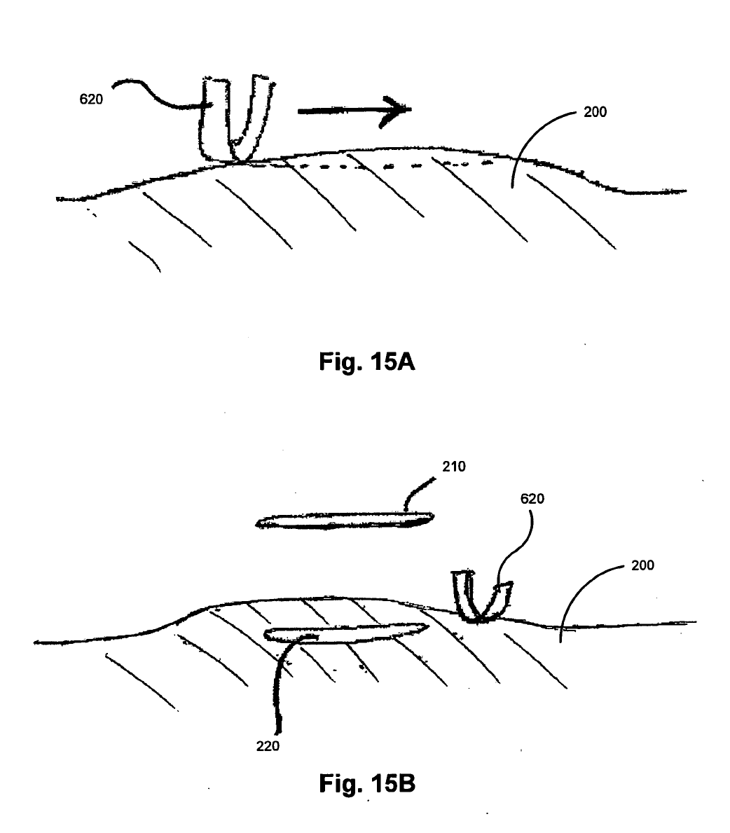

One common need in applications such as microsurgery is an incision of a predetermined length and depth. FIGS. 14A through 14D illustrate an embodiment of a device that can provide this type of controlled cut. As illustrated in FIG. 14A, the device comprises a housing 510, which surrounds a chamber 530 that is in communication with a vacuum port 540. A microknife 520 is mounted within the chamber 530 between an interior bellows 560 and an exterior bellows 570. The device further includes a depth stop 550 for controlling the depth of the cut made by the device. An internal orifice 565 allows for air flow between the interior bellows 560 and the chamber 530, while an exterior orifice 575 allows for air flow between the exterior bellows 570 and outside the device. In this way, the pressure within the interior bellows 560 will follow the pressure in the chamber 530, and the pressure in the exterior bellows 570 will follow the atmospheric pressure outside of the device. In FIG. 14A the device is shown with no vacuum applied, so all of the components are in their normal, unstressed state.

In FIG. 14B a vacuum is applied to the chamber 530. When this happens, the pressure inside the chamber 530 reaches the lowered vacuum pressure P quickly due to the large size of the vacuum connector 540. However, the internal orifice 565 provides a pinhole leak to the internal bellows 560, so the pressure within the bellows 560 begins to approach the chamber's pressure but cannot do so instantaneously. As the air leaks out of the internal bellows 560, the external bellows 570 expands and receives air through the external orifice 575 so that it can maintain equilibrium with the atmosphere. This causes a contraction of the internal bellows 560 and an expansion of the external bellows 570. As shown in FIG. 14C, this movement causes the knife 520 fixed between the internal bellows 560 and external bellows 570 to move from left to right in the drawings.

In addition to causing actuation of the knife 520, the vacuum applied within the chamber 530 causes the tissue 200 over which the device is place to lift up into the device chamber 530, as with the embodiments described above. Accordingly, when the knife 520 is moved due to the movement of the bellows 560 and 570 and the tissue is pulled up into the cutting path of the knife 520, a slicing incision is produced in the tissue. This slicing cut is continued until the bellows 560 and 570 reach equilibrium.

Once the cut is completed, as shown in FIG. 14D, the vacuum is turned off and the chamber 530 quickly returns to atmospheric pressure. The process described above is then reversed so that the knife is returned slowly to its unstressed position. Also when the vacuum is turned off, the tissue 200 elastically returns to its unstressed state so the knife 520 does not contact it on the knife's return stroke. This is because the bellows 560 and 570 return to their unstressed state slowly due to the flow restricting orifices 565 and 575. As air leaks into the internal bellows 560 and out of the external bellows 570, the device returns to the state shown in FIG. 14A. The result is a cut having a precise depth and length, where the depth is controlled by the depth stop 550 and the length is controlled in part by the vacuum pressure applied to the chamber 530. If a longer incision is desired, the device can be moved across the tissue in the direction of the desired cut and the above sequence repeated.

It is noted that the bellows 560 and 570 are not infinitely stiff, so they would be expected to sag; however, this sag may be desirable because it increases the force perpendicular to the tissue and the length of the stroke over which the knife contacts the tissue. In one embodiment, the bellows 560 and 570 comprise disposable plastic bellows that are made by molding, as is currently done in the manufacture of plastic and elastomeric bellows.

FIGS. 15A and 15B illustrate a modification of the technique described above in FIGS. 14A through 14D. In FIGS. 15A and 15B, a vacuum pressure is applied to an area of tissue 200 (the device not shown), and a three-dimensional curved microknife 620 is passed over the raised tissue. This movement causes the knife 620 to cut a strip 210 of tissue 200 that has been pulled into the knife's path. FIG. 15B illustrates the small strip 210 of tissue that has been severed and an underlying layer of tissue 220 that has been exposed. As described above, the vacuum pressure may then be turned off and the knife 620 returned to its original position. For example, the vacuum can be turned off, the device taken out of the way, and the severed strip 210 can removed, e.g., by tweezers. Various embodiments of curved microknives are described in related international application entitled “Three-Dimensional Cutting Instrument,” to Christopher Guild Keller, filed Aug. 13, 2007, which is incorporated by reference in its entirety.

In one embodiment, this procedure is performed using a device such as that described in FIGS. 14A through 14D, where the straight knife 520 is replaced with curved knife 620; however, other embodiments of the cutting instrument may be used, wherein a vacuum is applied and the knife 620 is passed over the raised tissue. In the embodiment illustrated, the knife 620 comprises a U-shaped blade.

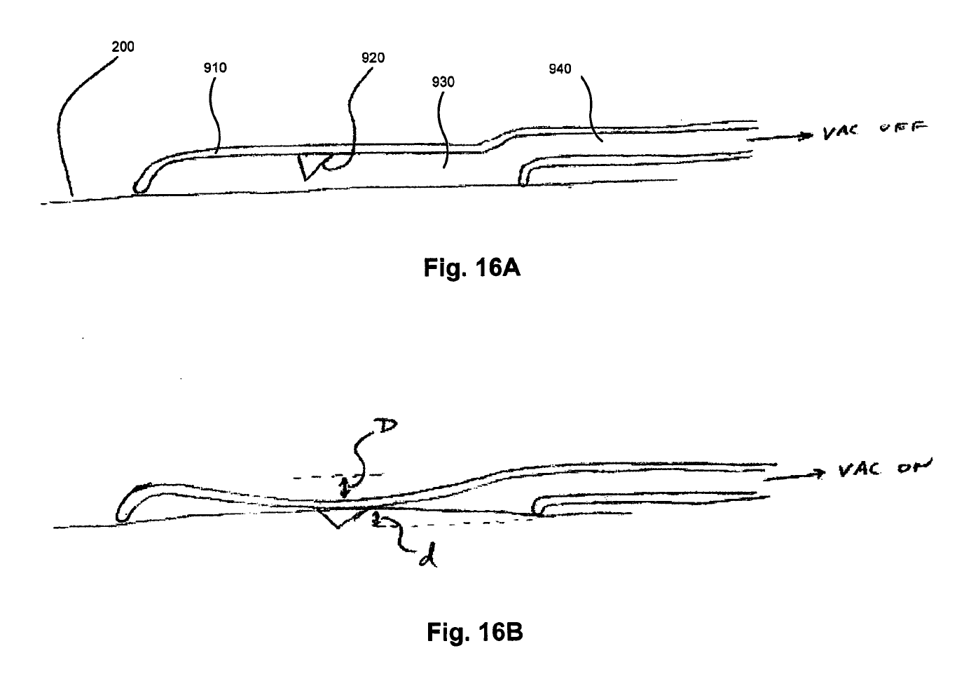

FIG. 16A is a cross sectional side view of a microscale cutting device in which a knife 920 is mounted in an vacuum chamber 930 of the device. A housing 910 of the device has a geometry that allows the open face of the chamber to seal upon contact with target tissue structure, where a vacuum port 940 couples the chamber 930 to a vacuum source for creating a vacuum pressure therein. FIG. 16B illustrates the device under application of the vacuum pressure. The housing 910 comprises a resilient material and has a suitable geometry (e.g., is sufficiently thin) to allow the knife 920, which is mounted to the housing 910, to deflect. When a sufficient vacuum pressure is applied, the knife 920 deflects by a distance D to contact the tissue, which itself deflects or bulges towards the knife 920 by a distance d. These opposing deflections produce an incision in the tissue.

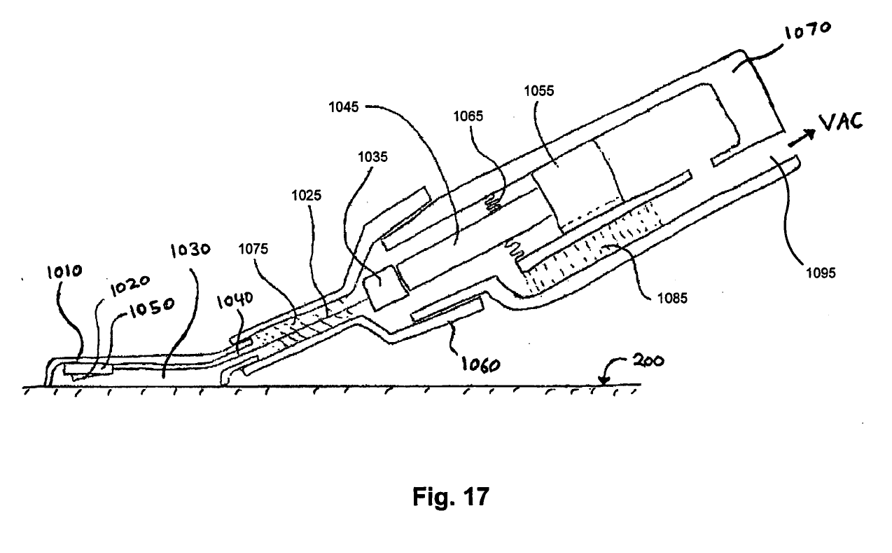

FIG. 17 is a cross sectional side view of a microscale cutting instrument, where a disposable or otherwise detachable cutting head (comprising, e.g., a housing 1010, microknife 1020, stop 1050, vacuum chamber 1030, and vacuum port 1040) is connected to a handle 1070. The handle 1070 contains a linear actuator 1055 that is operably coupled to the microknife 1020 by a wire 1025 or other connection mechanism suitable for moving the microknife 1020 across tissue 200. The action of moving the microknife 1020 across tissue 200 creates an incision longer than the width of the cutting edge of the microknife 1020. The linear actuator 1055 may comprise a solenoid, a motorized screw, or any other mechanism that can establish an attachment to and pull on the 1025 wire to move the microknife 1020.

The disposable or detachable portion of the device containing the microknife 1020 may be easily connected to and detached from the handle 1070 via a Luer lock needle 1060. The wire 1025 may be fixed to the microknife 1020 and detachably attached to the linear actuator 1055, for example, via a magnetically soft block 1035 (e.g., comprising a ferromagnetic material, such as a mu metal). When the disposable cutting head is attached to the handle 1070, the ferromagnetic block 1035 is brought into close proximity with a magnetic rod 1045 attached to the linear actuator 1055, which completes the mechanical coupling from the linear actuator 1055 to the rod 1045, to the block 1035, to the wire 1025, and ultimately to the microknife 1020. An elastomeric seal 1065 may be incorporated in the handle 1070, e.g., around the rod 1045, to separate the linear actuator 1055 and avoid contamination of the area of tissue 200 where the incision is being made. In alternative embodiments, mechanisms other than magnetic may be used to make this mechanical connection.

When the cutting head of the instrument is installed on the handle 1070, a pneumatic connection is made from a vacuum port 1095 of the handle to the vacuum chamber 1035 of the head. This allows the vacuum source to be attached to the handle 1070. In one embodiment, in the air flow path between the vacuum port 1095 and the chamber 1030, the device may comprise one or more filters 1075 and 1085. The filters 1075 and 1085 help to prevent debris and tissue material from being sucked into the handle 1070 and into the vacuum source when the vacuum is turned on.

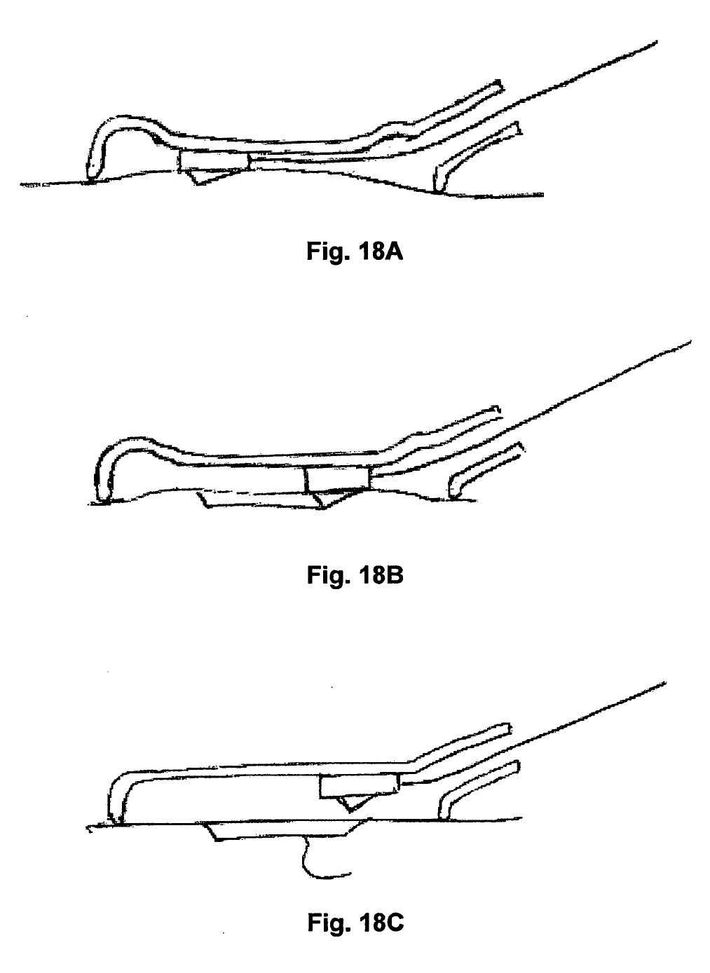

FIGS. 18A through 18C illustrate the operation of a cutting instrument, such as the one shown in FIG. 17. In a first step, shown in FIG. 18A, the vacuum is applied to the cutting device to bring the interior of the device to a low pressure. Under the vacuum pressure, the compliant roof of the device deflects downward toward the tissue, while the confronting tissue deflects upward toward the microknife. This causes the microknife to penetrate the tissue to a depth set by a depth stop on which the microknife is mounted. As illustrated in FIG. 18B, the linear actuator is activated to pull on the wire and move the microknife a predetermined distance. The knife is constrained vertically by the roof of the chamber and the tissue, and it is constrained laterally by side walls of the housing (not visible in centerline cross section), which may be straight or curved. The device may comprise more than one independently mounted microknives that are pulled by the linear actuator so that more than one straight or curved incisions may be made. Moreover, the microknife may comprise a three-dimensionally curved blade that cuts out a strip of tissue. When the procedure is complete, the vacuum pressure is turned off, and the microknife moves out of the tissue to stop the cut, as shown in FIG. 18C.

It is also noted that embodiments of the device will also work when partially or fully submerged in a low viscosity fluid such as water, blood, synovial fluid, cerebrospinal fluid, and the like. In such embodiments, a trap may be incorporated before the vacuum pump to gather the liquids sucked into the device. In addition, the chamber of the device may be vented with water or air as appropriate for a particular application.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure. The language used is in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A microscale cutting device comprising:
a housing defining a vacuum chamber within the housing;
a microknife mounted within the vacuum chamber of the housing;
a vacuum connector in communication with the vacuum chamber and adapted to be coupled to a vacuum source for creating a vacuum pressure within the housing.

2. The device of claim 1, further comprising:
a depth stop fixed to the device in relation to the microknife, the depth stop preventing a cut by the microknife beyond a predetermined maximum depth.

3. The device of claim 1, wherein the housing is shaped to fit over a planar tissue surface to enclose the vacuum chamber.

4. The device of claim 1, wherein the housing is shaped to fit over a cylindrical tissue surface to enclose the vacuum chamber.

5. The device of claim 1, wherein the housing is shaped to fit over a spherical tissue surface to enclose the vacuum chamber.

6. The device of claim 1, wherein the housing is shaped to fit over a concave tissue surface to enclose the vacuum chamber.

7. The device of claim 1, wherein the housing is shaped to fit over an irregular tissue surface to enclose the vacuum chamber.

8. The device of claim 1, wherein the housing is shaped to fit within a tubular structure to enclose the vacuum chamber.

9. The device of claim 1, wherein the microknife is coupled to an actuator configured to move the microknife towards a tissue surface when the housing is placed over tissue, thereby making a stab cut into the tissue.

10. The device of claim 1, wherein the microknife is coupled to an actuator configured to move the microknife in a direction transverse to a tissue surface when the housing is placed over tissue, thereby making a slice cut in the tissue.

11. The device of claim 10, wherein the microknife is curved for making a strip cut through a section of tissue.

12. The device of claim 1, further comprising:
an air valve pneumatically coupling the vacuum chamber to an ambient atmosphere;
a vacuum valve pneumatically coupling the vacuum chamber to a vacuum source; and
a valve controller operably coupled to open and close individually the air valve and the vacuum valve.

13. The device of claim 1, further comprising:
an internal pneumatic actuator coupled to the vacuum chamber; and
an external pneumatic actuator coupled to an atmosphere outside the vacuum chamber;
where the internal pneumatic actuator and external pneumatic actuator are coupled to opposing side of the vacuum chamber and the microknife coupled therebetween, wherein a lower pressure applied to the vacuum chamber relative to an atmospheric pressure causes contraction of the internal pneumatic actuator, expansion of the external pneumatic actuator, and resulting translation of the microknife.

14. The device of claim 13, wherein the internal pneumatic actuator and the external pneumatic actuator each comprise a bellows.

15. The device of claim 1, further comprising:
a pneumatic actuator coupling the microknife within the vacuum chamber, wherein a lower pressure applied to the vacuum chamber relative to an atmospheric pressure causes expansion of the pneumatic actuator and translation of the microknife.

16. The device of claim 1, wherein the housing is compliant such that a lower pressure applied to the vacuum chamber relative to an atmospheric pressure causes a deflection of the housing and translation of the microknife.

17. The device of claim 1, further comprising:
a handle; and
a linear actuator operably coupled to the microknife for pulling the microknife over a length of tissue.

18. The device of claim 1, further comprising:
a vacuum source coupled to the vacuum connector of the microscale cutting device and configured to create a vacuum pressure within the housing.

19. A device for performing a microscale operation on tissue, the device comprising:
a housing defining a vacuum chamber within the housing and configured to be placed over an area of tissue;
a vacuum connector in communication with the vacuum chamber and adapted to be coupled to a vacuum source for creating a vacuum pressure within the housing; and
a surgical tool mounted within the vacuum chamber of the housing.

20. The device of claim 19 wherein the surgical tool is mounted to the housing by a pneumatic actuator so that, upon application of a vacuum pressure within the housing, the pneumatic actuator expands to move the surgical tool.

21. The device of claim 20, wherein the pneumatic actuator comprises a bellows.

22. The device of claim 19, wherein the surgical tool is a needle.

23. The device of claim 22, further comprising:
a liquid-filled capsule in communication with the needle.

24. The device of claim 23, wherein the liquid-filled capsule is filled with a therapeutic agent.

25. The device of claim 23, further comprising:
a pneumatic actuator coupled between the housing and the needle, wherein upon application of a vacuum pressure within the housing when the device is placed adjacent to an area of tissue, the pneumatic actuator is configured to move the needle into the tissue and press against the liquid-filled capsule to force the liquid through the needle and into the tissue.

26. The device of claim 22, further comprising:
a guiding collar mounted around the needle to constrain the motion of the needle in one or more dimensions.

27. The device of claim 22, wherein the device is mounted within a catheter to allow injection from within a tubular structure.

28. A method for performing microscale cutting, the method comprising:
placing a housing of a microscale cutting device against an area of tissue to be treated to create a vacuum seal with the tissue, wherein the cutting device comprises a microknife mounted within the housing;
reducing the pressure within the housing of the microscale cutting device relative to outside the housing, thereby causing a portion of the tissue to tend to be forced toward the housing; and
cutting into the tissue with the microknife.

29. The method of claim 28, wherein the cutting comprises maintaining a consistent depth of cut using a depth stop fixed to the device in relation to the microknife.

30. The method of claim 28, wherein the area of tissue is planar and the housing is shaped to fit thereover to form the vacuum seal.

31. The method of claim 28, wherein the area of tissue is cylindrical and the housing is shaped to fit thereover to form the vacuum seal.

32. The method of claim 28 wherein the area of tissue is spherical and the housing is shaped to fit thereover to form the vacuum seal.

33. The method of claim 28, wherein the housing is shaped to fit over a concave tissue surface to enclose the vacuum chamber.

34. The method of claim 28, wherein the housing is shaped to fit over an irregular tissue surface to enclose the vacuum chamber.

35. The method of claim 28, wherein the housing is shaped to fit within a tubular structure to enclose the vacuum chamber.

36. The method of claim 28, wherein the cutting comprises:
actuating the microknife in an alternating fashion into and out of the tissue to create a series of stab cuts in the tissue; and
moving the microscale cutting device along the tissue.

37. The method of claim 36, wherein the microscale cutting device is moved at a rate no greater than half of the width of a single stab for each stab cut made.

38. The method of claim 28, wherein the cutting comprises:
raising the tissue surface by reducing the pressure within the housing; and
actuating the microknife in a direction transverse to the tissue surface and through a section of the tissue to create a slice cut in the tissue.

39. The method of claim 38, wherein the microknife is curved for making a strip cut through a section of tissue.

40. The method of claim 38, wherein actuating the microknife in a direction transverse to the tissue surface comprises:
contracting an internal pneumatic actuator and expanding an external pneumatic actuator by reducing the pressure in the housing, where the internal pneumatic actuator and external pneumatic actuator are coupled to opposing side of the vacuum chamber and the microknife coupled therebetween.

41. The method of claim 40, wherein the external pneumatic actuator and the internal pneumatic actuator each comprise a bellows.

42. The method of claim 28, wherein the cutting comprises:
expanding a pneumatic actuator by reducing the pressure in the housing to cause translation of the microknife.

43. The method of claim 42, wherein the pneumatic actuator comprises a bellows.

44. The method of claim 28, wherein the cutting comprises:
deflecting the housing of the device by reducing the pressure in the housing, where the deflection of the housing causes translation of the microknife.

45. The method of claim 28, wherein the cutting comprises:
move the microknife into the tissue; and
pulling the microknife over a length of tissue.

46. The method of claim 45, wherein the pulling the microknife comprises:
activating a linear actuator operably coupled to the microknife to pull thereon.
